(19) United States
(12) Patent Application Publication
(10) Pub. No.: US 2021/0093768 A1
(43) Pub. Date: Apr. 1, 2021

(54) HIGHLY-PERMEABLE DENSE HOLLOW FIBER MEMBRANE FOR BLOOD OXYGENATION

(71) Applicant: Tsinghua University, Beijing (CN)

(72) Inventors: Jisong ZHANG, Beijing (CN); Caijin ZHOU, Beijing (CN); Bingqi XIE, Beijing (CN)

(21) Appl. No.: 16/937,221

(22) Filed: Jul. 23, 2020

(30) Foreign Application Priority Data
Sep. 26, 2019 (CN) 201910915532.0

(51) Int. Cl. A61M 1/16; B01D 69/02; B01D 69/08; B01D 71/36; B01D 67/00

(52) U.S. Cl. CPC A61M 1/1621; B01D 69/02; B01D 69/08; B01D 71/36; B01D 2325/023; B01D 67/002; B01D 2325/38; B01D 2325/04; B01D 67/0013

(57) ABSTRACT

The present invention provides a highly-permeable dense hollow fiber membrane (HFM) for blood oxygenation. A membrane material plays a key role in an oxygenator, which determines the oxygenation efficiency, service life and safety of the oxygenator. The HFM according to the present invention features high permeability. When blood rich in carbon dioxide flows through the oxygenator, the carbon dioxide and oxygen in the blood can be rapidly exchanged, so that the blood can be rapidly updated, and the size of the oxygenator and the blood perfusion volume can be reduced. In addition, the membrane surface of the present invention is hydrophobic and dense, and blood does not directly contact with gas or permeate into a gas pipeline, thus avoiding the problems of protein leakage, permeability reduction and the like. The oxygenator prepared by using the HFM of the present invention can be repeatedly used for a long time.

### TECHNICAL FIELD

The present invention belongs to the technical field of biomedical engineering and particularly relates to a novel highly-permeable dense hollow fiber membrane (HFM) for blood oxygenation.

### BACKGROUND

Extracorporeal membrane oxygenation (ECMO), as an effective cardiopulmonary support therapy technique, is widely used in the treatment of severe acute cardiopulmonary failure and cardiovascular diseases, major surgeries, etc. An extracorporeal membrane oxygenator alternatively provides cardiopulmonary functions and spares more time for treating patients. Therefore, the extracorporeal membrane oxygenator represents the first-aid level of a country and hospitals.

The extracorporeal membrane oxygenator is essentially composed of a blood pump, an artificial lung and a gas source, where the artificial lung is the most important component. When blood is transported through the artificial lung by the blood pump, CO2 gas in the blood is quickly replaced by oxygen, so that the blood is updated. The membrane material is a key part in the process of gas replacement, and the membrane permeability thereof determines the gas exchange rate, and the surface of the material affects the physical, chemical, and biological properties of blood. At the beginning of the application of the membrane oxygenator, commonly used membrane materials are silicon and polypropylene, which have the advantages of fast gas exchange rate, etc. However, in practical application, it is found that the materials have poor biocompatibility and there are a lot of micropores on the membrane surface. In the oxygenation process, gas is in direct contact with blood through the micropores, blood cells are prone to hemolysis and other phenomena, causing thrombosis and other problems, thus increasing the risk of the blood oxygenation process. In addition, the membrane surface easily adsorbs blood proteins and platelets, leading to the deposition of the platelets and blocking the micropores on membrane surface, reducing oxygenation efficiency and safety, and greatly shortening the effective use time of the oxygenator. As the HFM technology is developed, polymethylpentene (PMP) is currently the most commonly used membrane material for oxygenators, which has the advantages of higher permeability and easiness of surface coating, etc., and can prolong oxygenation time to twenty eight days. However, due to the existence of the microporous structure, as the oxygenation time increases, there are still new problems such as the decrease of oxygenation efficiency and thrombosis. In addition, there is also a class of membrane material-based in vivo oxygenators used to assist a respiratory system and provide respiratory support for infants, which also has the problems of low oxygenation efficiency, short use time, poor safety and the like caused by membrane material problems. Therefore, it is of great significance to develop a membrane oxygenator based on a novel membrane material with a fast gas mass transfer rate and optimal biocompatibility to implement the efficient, safe and long-cycle blood oxygenation process.

### SUMMARY

The present invention overcomes the shortcomings of the prior art and provides a novel highly-permeable dense HFM for blood oxygenation, and the membrane material features optimal hydrophobicity and biocompatibility. In the oxygenation process, the dense HFM enables gas to rapidly pass through the membrane material by the principle of gas dissolution and diffusion, avoids direct contact between gas and blood while having the permeability similar to that of a conventional porous membrane material, and avoids the reduction of oxygenation efficiency caused by leakage of proteins and platelets; meanwhile, the surface of the membrane material has high hydrophobicity and biocompatibility, and can avoid blood coagulation while not coating anticoagulant substances, thereby ensuring the stability and oxygenation efficiency of the membrane material after long-term operation.

The foregoing objective of the present invention is implemented by the following technical solution:

A highly-permeable dense HFM for extracorporeal blood oxygenation is provided, where the membrane is formed by a hot melt extrusion forming method and a solution casting method, and features high permeability and hydrophobicity and optimal biocompatibility. The high permeability of the membrane material can improve the efficiency of blood-gas exchange. At the same time, unlike the prior art, in the oxygenation process, the membrane material only allows gas to pass through but does not allow blood to permeate, so that the gas exchange process is completed without direct contact between oxygen and blood, thus reducing the problems of blood cells injury, protein leakage, platelet adhesion and the like, and being beneficial to realizing an efficient and safe blood oxygenation process.

Further, a device essentially composed of a blood pump 1, a gas mass flow meter 2, an oxygenator 3, and a thermostatic water bath 4, and is used to verify the optimal membrane length, membrane thickness, and inner diameter of a hollow fiber of the HFM, and to characterize the stability and oxygenation efficiency of the HFM after long-term operation.

Further, the HFM is preferably made of Teflon AF 2400 and Teflon AF1600 materials, and the HFM is a dense membrane with a pore diameter of 0.01-0.1 nm. A hollow fiber of the HFM has an inner diameter of 20-500 and the HFM has a thickness of 5-100 μm and a length of 0.01-1 m. The HFM forming methods are preferably a melt extrusion forming method and a solvent casting method.

Further, the HFM is applied to an in vivo oxygenation process or an extracorporeal blood oxygenation process.

The present invention has the following beneficial effects as compared with the prior art.
1. Micropores on the surface of the membrane material adopted in the prior art can easily damage blood cells and lead to coagulation, thrombosis and other problems when used for a long time. The HFM of the present invention has a highly dense surface and does not have a micropore structure, thus avoiding direct contact between blood and oxygen, and reducing protein leakage, thrombosis and other problems.
2. After the currently used membrane material is used for long-term oxygenation, platelets and proteins adsorbed on the membrane surface permeate into and block the membrane pores, so that the stability and oxygenation efficiency of the membrane material are reduced. Compared with the membrane material, the HFM of the present invention does not allow liquid to permeate, and the surface of the material has high hydrophobicity, thus solving the problem of platelets and proteins adhering to the membrane surface, and ensuring the stability and efficiency of the membrane material after long-term oxygenation.
3. The HFM of the present invention has high permeability, and an oxygenator developed based on the membrane features high gas-liquid mass transfer efficiency, small size, small blood perfusion volume required before the oxygenation process, etc.

### BRIEF DESCRIPTION OF THE DRAWINGS

FIG. 1 is a schematic diagram of a characterization device for a membrane material oxygenation rate in Example 1 of the present invention; and

FIG. 2 is a microscopic view of a single hollow fiber of a HFM in Example 1 of the present invention.

### DETAILED DESCRIPTION

A material and miniature device for extracorporeal blood oxygenation according to the present invention will be described and illustrated in detail below with specific examples. In order to enable technicians to better understand the present invention, the examples cannot be understood as limiting the protection scope of the present invention.

### Example 1

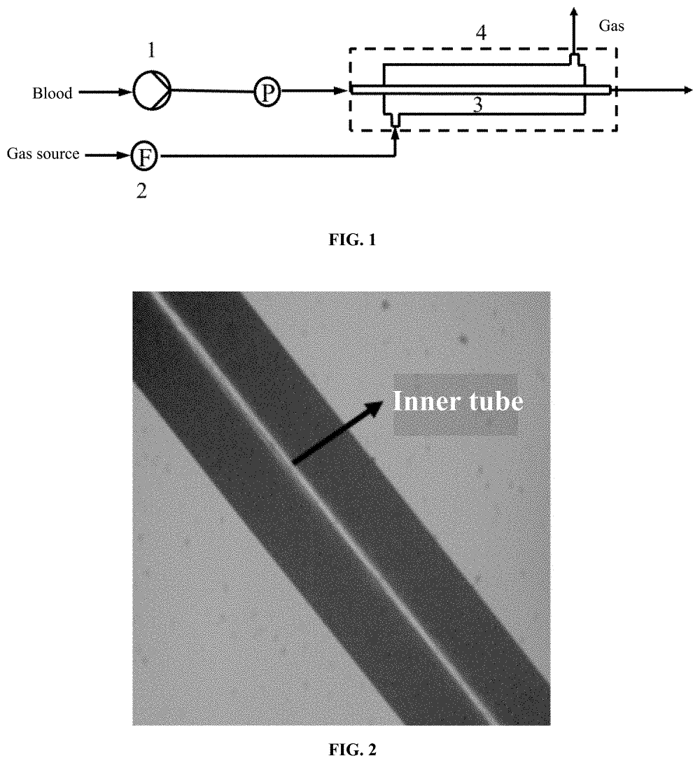

A device essentially composed of a blood pump 1, a gas mass flow meter 2, an oxygenator 3, a thermostatic water bath 4 is provided. As shown in FIG. 1, the thickness and length of HFM and the diameter of the hollow fiber shown in FIG. 2 were optimized to achieve the optimal oxygenation efficiency. Specific implementation steps are as follows.
(1) Blood rich in carbon dioxide was continuously injected into an inner tube of an oxygenator 3 through the blood pump 1; at the same time, oxygen was continuously introduced into an outer tube, and the oxygen flow rate was controlled by the gas mass flow meter 2; the blood flow rate was regulated by the blood pump, the blood flow rate was constant at 2 ml/min, the oxygen flow rate was 4 ml/min, the blood oxygenation process was kept at 37° C., and the pressure drop of a blood pipeline was measured by a micro pressure sensor.
(2) A small amount of blood was taken from an outlet of the oxygenator, the oxygen concentration was measured through a blood-gas analyzer, and the oxygenation efficiency of the HFM material was analyzed under different implementation conditions.
(3) The length of the HFM was kept at 0.4 m and the inner diameter of a hollow fiber was 200 um, and when the thicknesses of the HFM were 20 um, 40 um, 60 um, 80 um and 100 um respectively, the oxygenation rates of the dense HFM were 0.188, 0.175, 0.123, 0.0101 and 0.007 mL/min respectively.
(4) The thickness of the HFM was kept at 40 um and the inner diameter of the hollow fiber was 200 μm, and when the lengths of the HFM were 0.1 m, 0.2 m, 0.4 m, 0.6 m and 0.8 m respectively, the oxygenation rates of the dense HFM were 0.08, 0.14, 0.18, 0.184 and 0.188 mL/min respectively.
(5) The thickness of the HFM was kept at 40 μm and the length was 0.4 m, and when the inner diameters of the hollow fiber were 50 μm, 100 μm, 200 μm, 400 μm and 500 μm respectively, the oxygenation rates of the dense HFM were 0.189, 0.181, 0.175, 0.121 and 0.081 mL/min respectively.

### Example 2

A device essentially composed of a blood pump 1, a gas mass flow meter 2, an oxygenator 3, a thermostatic water bath 4 is provided. As shown in FIG. 2, a microscopic view of a single hollow fiber of an HFM is characterized. Specific implementation steps are as follows.
(1) Blood rich in carbon dioxide was continuously injected into an inner tube of an oxygenator through the blood pump; at the same time, oxygen was continuously introduced into an outer tube, and the oxygen flow rate was controlled by the gas mass flow meter; the blood flow rate was regulated by the blood pump, the blood flow rate was constant at 2 ml/min, the oxygen flow rate was 4 ml/min, the blood oxygenation process was kept at 37° C., and the pressure drop of a blood pipeline was measured by a micro pressure sensor. The HFM had a length of 0.4 m and a thickness of 40 μm, and the hollow fiber has an inner diameter of 200 μm.
(2) A small amount of blood was taken from an outlet of the oxygenator, the oxygen concentration was measured through a blood-gas analyzer, and the oxygenation efficiency of the HFM material under different oxygenation time was analyzed. The obtained results are shown in Table 1.

Table 1 shows the change of oxygenation rate of the HFM under different oxygenation time in Example 2 of the present invention.

TABLE 1

| Oxygenation time (day) | Oxygenation rate (mL/min) |
|---|---|
| 1 | 0.173 |
| 5 | 0.175 |
| 10 | 0.176 |
| 20 | 0.173 |
| 40 | 0.178 |
| 60 | 0.175 |

The technical solution of the present invention has been described in detail by the foregoing examples. Obviously, the present invention is not limited to the described examples. Based on the examples of the present invention, those skilled in the art can also make various changes accordingly, and any changes equivalent to or similar to the present invention fall within the protection scope of the present invention.

1. A highly-permeable dense hollow fiber membrane (HFM) for blood oxygenation, wherein the membrane is prepared from Teflon AF 2400 and Teflon AF1600 materials, and features high permeability, desirable hydrophobicity and optimal biocompatibility.

2. The HFM according to claim 1, wherein the HFM is a dense membrane with a pore diameter of 0.01-0.1 nm.

3. The HFM according to claim 1, wherein the HFM has a thickness of 5-100 um.

4. The HFM according to claim 1, wherein a hollow fiber of the HFM has an inner diameter of 20-500 μm.

5. The HFM according to claim 1, wherein the HFM has a length of 0.01-1 m.

6. The HFM according to claim 1, wherein the HFM is formed by a melt extrusion forming method or a solvent casting method.

7. The HFM according to claim 1, wherein the HFM is applied to an in vivo oxygenation process or an extracorporeal blood oxygenation process.
